(19) United States
(12) Patent Application Publication
(10) Pub. No.: US 2020/0347419 A1
(43) Pub. Date: Nov. 5, 2020

(54) RECOMBINANT BACTERIUM CAPABLE OF PRODUCING L-LYSINE, CONSTRUCTION METHOD THEREOF AND PRODUCTION METHOD OF L-LYSINE

(71) Applicants: Ningxia Eppen Biotech Co., Ltd, Yin Chuan, Ning Xia (CN); Institute of Microbiology, Chinese Academy of Sciences, Chaoyang District, Beijing (CN)

(72) Inventors: Tingyi WEN, Chaoyang District, Beijing (CN); Chen ZHANG, Chaoyang District, Beijing (CN); Xiuling SHANG, Chaoyang District, Beijing (CN); Xin CHAI, Chaoyang District, Beijing (CN); Yun ZHANG, Chaoyang District, Beijing (CN); Shuwen LIU, Chaoyang District, Beijing (CN); Guoqiang WANG, Chaoyang District, Beijing (CN); Zhongcai LI, Chaoyang District, Beijing (CN)

(21) Appl. No.: 16/760,662

(22) PCT Filed: May 22, 2018
(86) PCT No.: PCT/CN2018/087894

§ 371 Date: Apr. 30, 2020

(30) Foreign Application Priority Data
Nov. 1, 2017 (CN) 201711058221.4

(51) Int. Cl. C12P 13/08; C12N 15/64; C12N 15/69

(52) U.S. Cl. CPC C12P 13/08; C12R 1/15; C12N 15/69; C12N 15/64

(57) ABSTRACT

A recombinant bacterium for producing L-lysine, a construction method thereof, and a method for producing L-lysine by using the recombinant bacterium. The recombinant bacterium has increased expression and/or activity of asparaginase compared to a starting bacterium.

### TECHNICAL FIELD

The present invention generally relates to the field of microbial fermentation, and specifically relates to a recombinant bacterium capable of producing L-lysine, a construction method thereof, and a production method of L-lysine.

### BACKGROUND

L-lysine is one of the nine essential amino acids of the human body. It has various physiological functions such as regulating the body's metabolic balance and promoting growth and development. It is widely used in the fields of food, feed and medicine. In the feed industry, lysine is the first limiting amino acid for the growth of pigs and poultry. Adding L-lysine to the feed can improve the utilization rate of amino acids and proteins in the feed, improve the nutritional potency of the feed, and promote the growth of livestock and poultry. In the food industry, L-lysine is mainly used for nutrition enhancers and deodorants. In the field of medicine, L-lysine is one of the main components of compound amino acid preparations. At present, the lysine industry is the second largest amino acid industry after glutamic acid. Therefore, the industrial production research of L-lysine is of great significance.

At present, L-lysine is mainly produced by direct fermentation of microorganisms. The fermentation performance of lysine-producing bacteria is a key factor affecting the production cost of the fermentation method.

The breeding methods of high-producing strains of lysine mainly include traditional mutagenesis and metabolic engineering transformation.

The strains obtained through mutagenesis screening will accumulate a large number of negative-effect mutations, resulting in problems such as slow growth of the strains, reduced environmental tolerance and increased nutritional requirements. These defects limit the industrial application of strains.

As shown in FIG. 1, in the anabolic pathway of lysine from Corynebacterium glutamicum, the synthetic precursor of lysine is oxaloacetic acid in the tricarboxylic acid cycle (TCA cycle). The oxaloacetic acid is converted into aspartic acid through transamination to enter the synthesis pathway of lysine. Therefore, the metabolic engineering transformation of lysine-producing strains in the prior art mainly focuses on the terminal synthesis pathway of lysine, the glycolysis pathway that provides synthetic precursors, the TCA cycle, and the modification of key genes in the pentose phosphate pathway that provides the cofactor NADPH. Specifically, it mainly increases the synthesis of oxaloacetate by enhancing the expression of pyruvate carboxylase gene (pyc gene) and weakening the expression of phosphoenolpyruvate carboxykinase gene (pck gene), so as to increase the accumulation of lysine. However, to date, there is no existing technology for metabolic engineering transformation of lysine-producing strains from the perspective of affecting the supply of aspartic acid.

### SUMMARY

The inventor discovered in the previous research that the supply of aspartic acid is also a key factor affecting the synthesis of lysine. Increasing the synthesis of aspartic acid can ensure the supply of precursors for massive synthesis of lysine and increase the lysine synthesis efficiency of strains. In the metabolism process of aspartic acid, aspartic acid is catalyzed by asparagine synthase to produce asparagine, and asparagine is catalyzed by asparaginase to produce aspartic acid and ammonia.

The purpose of the present invention is to provide a recombinant bacterium capable of producing L-lysine by carrying out metabolic engineering modification of lysine-producing strains.

The present invention provides a recombinant bacterium capable of producing L-lysine, wherein the recombinant bacterium has increased expression and/or activity of asparaginase (EC 3.5.1.1 asparaginase) compared to an original bacterium. The original bacterium refers to a strain capable of accumulating lysine.

Preferably, the recombinant bacterium according to the above description, wherein the recombinant bacterium has at least two copies of asparaginase encoding gene, and/or the expression of the asparaginase encoding gene of the recombinant bacterium is mediated by a regulatory element with high transcription or high expression activity. Preferably, the regulatory element is a strong promoter. More preferably, the strong promoter is a P_{tuf} promoter of the original bacterium.

Preferably, the recombinant bacterium according to the above description, wherein the recombinant bacterium has reduced expression and/or activity of homoserine dehydrogenase (Hom) compared to the original bacterium. The reduced homoserine dehydrogenase expression is achieved in at least one of the following ways: (A) the homoserine dehydrogenase encoding gene of the recombinant bacterium is inactivated, and (B) the expression of the homoserine dehydrogenase encoding gene of the recombinant bacterium is mediated by a regulatory element with low transcription or low expression activity. The reduced activity of homoserine dehydrogenase is achieved by mutating the 59th valine of the homoserine dehydrogenase of the recombinant bacterium to alanine, wherein, preferably, the homoserine dehydrogenase encoding gene of the recombinant bacterium is SEQ ID NO.1.

Preferably, the recombinant bacterium according to the above description, wherein the recombinant bacterium has increased expression and/or activity of pyruvate carboxylase (pyc) compared to the original bacterium. Preferably, the increased expression of pyruvate carboxylase is achieved in at least one of the following ways: (C) the recombinant bacterium has at least two copies of pyruvate carboxylase encoding gene, and (D) the expression of the pyruvate carboxylase encoding gene of the recombinant bacterium is mediated by a regulatory element with high transcription or high expression activity. The increased activity of pyruvate carboxylase is achieved by mutating the 458th proline of the pyruvate carboxylase of the recombinant bacterium to serine, wherein, preferably, the pyruvate carboxylase encoding gene of the recombinant bacterium is SEQ ID NO.8.

Preferably, the recombinant bacterium according to the above description, wherein the recombinant bacterium has reduced expression and/or activity of phosphoenolpyruvate carboxykinase (pck) compared to the original bacterium. Preferably, the phosphoenolpyruvate carboxykinase encoding gene of the recombinant bacteria is inactivated, and/or the expression of the phosphoenolpyruvate carboxykinase encoding gene is mediated by a regulatory element with low transcription or low expression activity. More preferably, the inactivation is implemented by knocking out the phosphoenolpyruvate carboxykinase encoding gene of the recombinant bacterium.

Preferably, the recombinant bacterium according to the above description, wherein the recombinant bacterium has increased expression and/or activity of dihydropyridine dicarboxylate reductase (dapB) compared to the original bacterium. Preferably, the recombinant bacterium has at least two copies of dihydropyridine dicarboxylate reductase encoding gene, and/or the expression of the dihydropyridine dicarboxylate reductase encoding gene is mediated by a regulatory element with high transcription or high expression activity. More preferably, the regulatory element is a strong promoter. Most preferably, the strong promoter is a P_{tuf} promoter of the original bacterium.

Preferably, the recombinant bacterium according to the above description, wherein the recombinant bacterium has increased expression and/or activity of aspartate kinase (ysC), diaminopimelate dehydrogenase (ddh) and/or diaminopimelate decarboxylase (ysA) compared to the original bacterium. Preferably, the recombinant bacterium has at least two copies of aspartate kinase encoding gene, diaminopimelate dehydrogenase encoding gene and/or diaminopimelate decarboxylase encoding gene, and/or the expression of the aspartate kinase encoding gene, the diaminopimelate dehydrogenase encoding gene and/or the diaminopimelate decarboxylase encoding gene is mediated by a regulatory element with high transcription or high expression activity. More preferably, the regulatory element is a strong promoter. Most preferably, the strong promoter is a P_{tuf} promoter of the original bacterium.

Or preferably, the recombinant bacterium according to the above description, wherein the original bacterium is a bacterium selected from Corynebacterium, Brevibacterium, Bacillus, Bifidobacterium, and Lactobacillus or a fungus selected from yeast.

The bacterium of Corynebacterium is selected from Corynebacterium glutamicum, Corynebacterium pekinense, Corynebacterium eficiens, Corynebacterium crenatum, Corynebacterium thermoaminogenes, Corynebacterium aminogenes, Corynebacterium lilium, Corynebacterium callunae, and Corynebacterium herculis.

The bacterium of Brevibacterium is selected from Brevibacteriaceae fivum, Brevibacteriaceae lactofermentum and Brevibacteriaceae ammoniagenes.

The bacterium of Bacillus is selected from Bacillus licheniformis, Bacillus subtilis or Bacillus pumilus.

The bacterium of Bifidobacterium is selected from Bifdobacterium bifidum, Bifidobacterium longum, Bifidobacterium breve, and Bifidobacterium adolescentis.

The bacterium of Lactobacillus is selected from Lactobacillus acidophilus, Lactobacillus casei, Lactobacillus delbrueckii subsp and Lactobacillus fermentum.

The fungus of yeast is selected from Candida utilis, Saccharomyces cerevisiae, Pichia pastoris or Hansenula polymorpha.

The present invention further provides a construction method of the above-mentioned recombinant bacterium, comprising the following step: increasing the expression and/or activity of asparaginase in a original bacterium. Specifically, increasing the expression and/or activity of the asparaginase in the original bacterium is achieved by at least one of the following ways: (E) increasing the copy number of asparaginase encoding gene in the original bacterium, and (F) replacing a regulatory element for the asparaginase encoding gene in the original bacterium with a regulatory element with high transcription or high expression activity.

Preferably, the construction method further comprises the step of reducing the expression and/or activity of homoserine dehydrogenase in the original bacterium.

Preferably, the construction method further comprises the step of increasing the expression and/or activity of pyruvate carboxylase in the original bacterium.

Preferably, the construction method further comprises the step of reducing the expression and/or activity of phosphoenolpyruvate carboxykinase in the original bacterium. Specifically, reducing the expression and/or activity of phosphoenolpyruvate carboxykinase in the original bacterium is achieved by at least one of the following ways: (G) inactivating, preferably knocking out, the phosphoenolpyruvate carboxykinase encoding gene in the chromosome of the original bacterium, and (H) replacing a regulatory element for the phosphoenolpyruvate carboxykinase encoding gene in the original bacterium with a regulatory element with low transcription or low expression activity.

Preferably, the construction method further comprises the step of increasing the expression and/or activity of dihydropyridine dicarboxylate reductase in the original bacterium. Specifically, increasing the expression and/or activity of the dihydropyridine dicarboxylate reductase in the original bacterium is achieved by at least one of the following ways: (I) increasing the copy number of dihydropyridine dicarboxylate reductase encoding gene in the original bacterium, and (J) replacing a regulatory element for the dihydropyridine dicarboxylate reductase in the original bacterium with a regulatory element with high transcription or high expression activity.

Or preferably, the construction method further comprises the step of increasing the expression and/or activity of aspartate kinase, diaminopimelate dehydrogenase and/or diaminoheptanoate decarboxylase in the original bacterium. Specifically, increasing the expression and/or activity of aspartate kinase, diaminopimelate dehydrogenase and/or diaminopimelate decarboxylase in the original bacterium is achieved by at least one of the following ways: (L) increasing the copy number of aspartate kinase encoding gene, diaminopimelate dehydrogenase encoding gene and/or diaminoheptanoate decarboxylase encoding gene in the original bacterium, and (M) replacing regulatory elements for the aspartate kinase encoding gene, the diaminopimelate dehydrogenase encoding gene and/or the diaminoheptanoate decarboxylase encoding gene with regulatory elements with high transcription or high expression activity.

The present invention further provides a production method of L-lysine, including the following step: fermenting and culturing the above recombinant bacterium.

Through fermentation culture, it is observed that the recombinant bacterium capable of producing L-lysine provided by the present invention has superposition effect of increasing the production, and significantly improve the production of L-lysine. The lysine production intensity after 48 h of fermentation is 0.05-5 g/L/h, and the lysine production at the end of fermentation is 1-300 g/L.

The present invention first provides a metabolic engineering strategy for increasing the supply of aspartic acid, which is a precursor of lysine synthesis, by enhancing the expression of asparaginase. It can significantly increase the production of lysine, and thus can be used in bacterial fermentation to produce lysine in practice. It has developed a new method able to increase the fermentation production of lysine. It is observed that the effect of the production increasing can be superimposed, so that it can be used in bacterial fermentation to produce lysine in practice, which is convenient for promotion and application.

### BRIEF DESCRIPTION OF THE DRAWINGS

FIG. 1 is a schematic diagram of the anabolic pathway of lysine from Corynebacterium glutamicum;

FIG. 2 is a schematic diagram of recombinant plasmid YZ022;

FIG. 3 is a schematic diagram of recombinant plasmid YZ023;

FIG. 4 is a schematic diagram of recombinant plasmid YZ025;

FIG. 5 is a schematic diagram of recombinant plasmid YE019;

FIG. 6 is a schematic diagram of recombinant plasmid YZ037;

FIG. 7 is a schematic diagram of recombinant plasmid YZ039; and

FIG. 8 is a schematic diagram of recombinant plasmid YZ035.

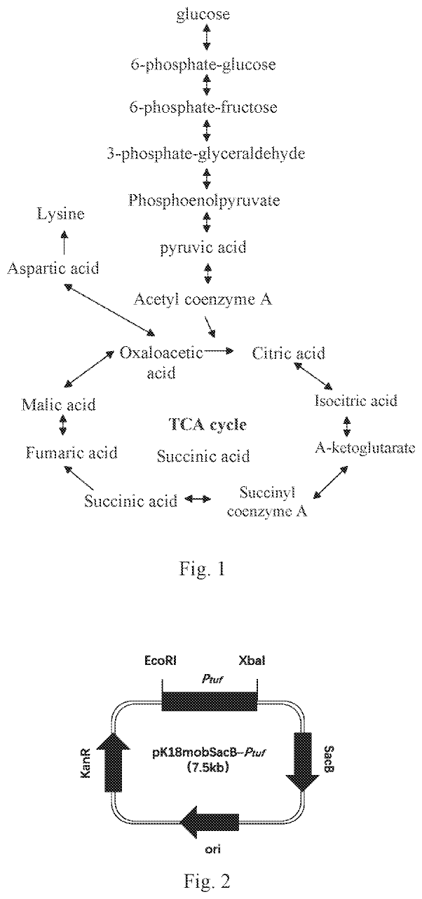

### DETAILED DESCRIPTION OF EMBODIMENTS

The embodiments of the present invention will be described in more detail in conjunction with the accompanying drawings and embodiments, in order to provide a better understanding of the embodiments of the present invention and the advantages thereof. However, the specific embodiments and examples described below are illustrative only and should not be construed as limiting the present invention.

The present invention relates to a recombinant bacterium capable of producing L-lysine, wherein the recombinant bacterium has increased expression and/or activity of asparaginase compared to a original bacterium. The original bacterium refers to a strain capable of accumulating lysine.

Increased expression and/or activity of asparaginase can be realized based on various factors, comprising increased copy number of the coding gene, replacement of the natural promoter with a more effective strong promoter, and artificial mutations intended to increase the activity. Specifically, the gene copy number can be increased by the introduction and/or amplification of endogenous and/or exogenous alleles. As for the replacement of gene promoters, its examples comprise the introduction of endogenous and/or exogenous promoters. The promoters used have effective activity to effectively enhance the expression of downstream structural genes.

In one embodiment, the recombinant bacterium has at least two copies of asparaginase encoding gene. Specifically, the recombinant bacterium has one or more copies of endogenous and/or exogenous asparaginase encoding gene in its nuclear DNA in addition to one copy of the endogenous asparaginase encoding gene. More specifically, the nucleotide sequence of the asparaginase encoding gene can be SEQ ID NO.39.

In one embodiment, the expression of the asparaginase encoding gene of the recombinant bacterium is mediated by a regulatory element with high transcription or high expression activity. Preferably, the regulatory element is a strong promoter. More preferably, the strong promoter is a P_{f}r promoter of the original bacterium. Specifically, at the upstream of the asparaginase encoding gene in the the nuclear DNA of the recombinant bacterium, there is an effective endogenous and/or exogenous strong promoter, resulting in an effective increase in the expression of the asparaginase encoding gene.

The “original strain” in the present invention refers to the initial strain used in the genetic modification strategy of the present invention. The strain may be a naturally occurring strain, or may be a strain bred by mutagenesis or genetic engineering.

The expression “inactivation” in the present invention refers to “inactivation” in the present invention refers to that the corresponding modified object changes to achieve a certain effect, including but not limited to, site-directed mutation, insertional inactivation and/or knockout.

The methods of gene knockout, gene insertion, promoter replacement and site-directed mutation described in the present invention can be realized by homologous recombination of a homologous arm with a modified target gene carried by a vector.

The introduction of a gene or the increase in the copy number of a gene according to the present invention can be achieved by constructing a recombinant plasmid containing the gene and then introducing the recombinant plasmid into the original bacterium, or by directly inserting a gene into a suitable site on the chromosome of the original bacterium.

Although examples of regulatory elements with high transcription or high expression activity are given in the present invention, the regulatory elements with high transcription or high expression activity are not particularly limited in the present invention, as long as they can enhance the expression of the promoter genes. The regulatory elements that can be used in the present invention comprise P₄₅, P_{eftu}, P_{sod}, P_{glyA}, P_{pck}, P_{pgk} promoters of the original bacterium, etc. but are not limited thereto. The regulatory elements with low transcription or low expression activity are also not particularly limited in the present invention, as long as they can reduce the expression of the gene to be promoted.

The experimental methods in the following embodiments are conventional methods unless otherwise specified. Unless otherwise specified, the materials and reagents used in the following embodiments can be commercially available.

Unless otherwise specified in the following embodiments, the technical means used in the embodiments are conventional means well known to those skilled in the art, see “Molecular Cloning: A Laboratory Manual (3rd Edition)” (Science Press), “Microbiology Experiment (4th Edition)” (Higher Education Press), the manufacturer's instructions for the corresponding instruments and reagents, etc. Instruments and reagents used in the embodiments are commonly used instruments and reagents in the market. For the quantitative tests in the following embodiments, three replicate experiments are set, and the results are averaged.

### Example 1 Construction of Lysine Chassis Engineering Bacterium

In this example, the site-directed mutation was performed on hom (homoserine dehydrogenase, GenBank: CAF19887.1) gene of the original strain Corynebacterium glutamicum wild-type ATCC13032 to reduce the metabolic flux of a branch pathway, i.e., the synthesis pathway of threonine; site-directed mutation was performed on pyc (Pyruvate carboxylase, GenBank: CAF19394.1) gene to increase the supply of oxaloacetate which is a synthesis precursor of lysine; knockout of pck (phosphoenolpyruvate carboxykinase, GenBank: CAF20888.1) gene was performed and the copy number of pyc* and dapB (dihydropyridine dicarboxylate reductase, GenBank: CAF20314.1) gene were also increased; IysC (aspartate kinase, GenBank: CAF18822.1), ddh (Diaminopimelate dehydrogenase, GenBank: CAF21279.1), and lysA (diaminoheptanoate decarboxylase, GenBank: CAF19884.1) genes of plasmids were overexpressed to further enhance the synthesis pathway of lysine to construct a lysine-producing chassis engineering bacterium.

(1) Site-Directed Mutation of Chromosome Hom Gene

Primers were designed respectively according to the hom gene of Corynebacterium glutamicum ATCC13032 in Genbank and its upstream and downstreamsequences.

Using the genomic DNA of Corynebacterium glutamicum ATCC13032 as a template, primers were designed at the mutation site to amplify two parts of the hom gene at upstream and downstream of the mutation site, respectively. The upper half of the hom gene was amplified with P1 and P2 as primers, and the lower half of the hom gene was amplified with P3 and P4 as primers. Then using the above purified PCR product as a template and P1 and P4 as primers, SOE (gene splicing by overlap extension) PCR was performed for amplification, to obtain 1638 bp PCR product, which contains hom gene (SEQ ID NO. 1) with the 59th valine mutated to alanine (V59A Mutation).

The above 1638 bp PCR product was double-digested with Xba I and EcoR I, and then ligated with the double-digested homologous recombinant vector pK18mobsacB (purchased from ATCC, Cat. No. 87097). The ligation product was transformed into E. coli DH5α by chemical transformation, and the transformants were screened on LB plates containing kanamycin (50 μg/mL). After the subculture for three generations, transformants were identified by colony PCR using P5 and P6 as primers; a plasmid was extracted from the transformant identified positive, and the plasmid was double digested by Xba I and EcoR I and identified; the obtained 1638 bp plasmid was positive.

The positive plasmid was sent for sequencing. As a result, the plasmid was a recombinant plasmid obtained by inserting the nucleotide shown in SEQ ID NO. 1 in the sequence table into the vector pK18mobsacB, and named YE019, shown in FIG. 5.

TABLE 1

| | | SEQ |
|---|---|---|
| | | ID |
| Primer | Base sequence | NO. |
| P1 | GCTCTAGAAGCTGTTTCACAATTTCT | 2 |
| P2 | ATATCAGAAGCAGCAATGC | 3 |
| P3 | GCATTGCTGCTTCTGATAT | 4 |
| P4 | CCGGAATTCCCAACAACTTGATGGTGT | 5 |
| P5 | TCTACGTTGTATCTCGCAC | 6 |
| P6 | CAGGCGACCAGCTGCTTC | 7 |

The homologous recombinant plasmid YE019 sequenced positive was electrotransformed into Corynebacterium glutamicum wild-type ATCC13032. Colonies with recombinant plasmids integrated into chromosomes were obtained by kanamycin resistance forward screening. Positive colonies with second homologous recombination were obtained by sucrose reverse screening. Using P5 and P6 as primers, PCR amplification and identification was carried out on the positive colonies to obtain a recombinant bacterium identified positive, named Corynebacterium glutamicum EPCG1000.

The genomic DNA of the recombinant bacterium was extracted and sequenced. The results confirmed that the hom gene in Corynebacterium glutamicum wild-type ATCC13032 had been successfully replaced with the hom gene at V59A, and Corynebacterium glutamicum EPCG1000 was successfully constructed.

(2) Site-Directed Mutation of Chromosome Pyc Gene

Primers were designed respectively according to the pyc gene of Corynebacterium glutamicum ATCC13032 in Genbank and its upstream and downstreamsequences.

Using the genomic DNA of Corynebacterium glutamicum ATCC13032 as a template, primers were designed at the mutation site to amplify two parts of the pyc gene at upstream and downstream of the mutation site, respectively. The upper half of the pyc gene was amplified with P7 and P8 as primers, and the lower half of the pyc gene was amplified with P9 and P10 as primers. Then using the purified PCR product as a template and P7 and P10 as primers, SOE (gene splicing by overlap extension) PCR was performed for amplification, to obtain 3423 bp PCR product, which was pyc gene (SEQ ID NO. 8) with the 458th proline mutated to alanine (P458S Mutation), i.e., pyc* gene.

The above 3423 bp PCR product was double-digested with Xba I and Hind III, and then ligated with the double-digested homologous recombinant vector pK18mobsacB (purchased from ATCC, Cat. No. 87097). The ligation product was transformed into E. coli DH5α by chemical transformation, and the transformants were screened on LB plates containing kanamycin (50 μg/mL). After the subculture for three generations, the transformants were identified by colony PCR using P11 and P12 as primers; a plasmid was extracted from the transformant identified positive, and the plasmid was double digested by Xba I and Hind III and identified; the obtained 3423 bp plasmid was positive.

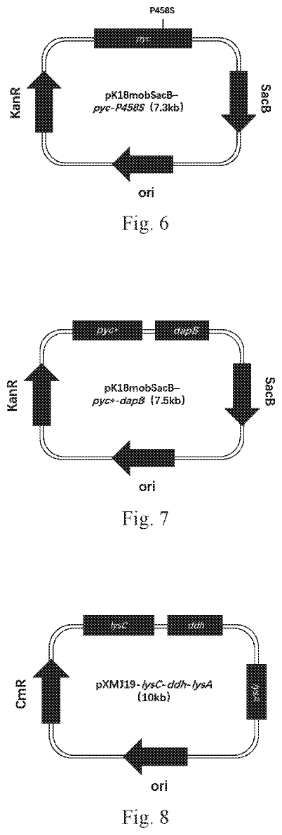

The positive plasmid was sent for sequencing. As a result, the plasmid was a recombinant plasmid obtained by inserting the nucleotide shown in SEQ ID NO. 8 in the sequence table into the vector pK18mobsacB, and named YZ037 shown in FIG. 6.

TABLE 2

| | | SEQ |
|---|---|---|
| | | ID |
| Primer | Base sequence | NO. |
| P7 | CCCAAGCTTTGACTGCTCACTGCAGCGT | 9 |
| P8 | AGGTGCGAGTGATCGGC | 10 |
| P9 | GCCGATCACTCGCACCT | 11 |
| P10 | GCTCTAGAGCGTCGATTGCTGGACGC | 12 |
| P11 | CGCAAATTAGCAACAGAAG | 13 |
| P12 | CCTTAATGGCCAAGATGT | 14 |

The homologous recombinant plasmid YZ037 sequenced positive was electrotransformed into Corynebacterium glutamicum EPCG1000. Colonies with recombinant plasmids integrated into chromosomes were obtained by kanamycin resistance forward screening. Positive colonies with second homologous recombination were obtained by sucrose reverse screening. Using P11 and P12 as primers, PCR amplification and identification was carried out on the positive colonies to obtain a recombinant bacterium identified positive, named Corynebacterium glutamicum EPCG1007.

The genomic DNA of the recombinant bacterium was extracted and sequenced. The results confirmed that the pyc gene in Corynebacterium glutamicum EPCG1000 had been successfully replaced with the pyc* gene having a mutation at P458S, and Corynebacterium glutamicum EPCG1007 was successfully constructed.

(3) Knockout of Pck Gene and Increase in Copies of Pyc*-dapB

Primers were designed respectively according to the pck gene of Corynebacterium glutamicum ATCC13032 in Genbank and its upstream and downstream sequences.

Using the genomic DNA of Corynebacterium glutamicum ATCC13032 as a template, the sequence (SEQ ID NO.15) of the upstream part of the pck gene was amplified with P13 and P14 as primers, the promoter of pyc gene was amplified with P15 and P16 as primers, the sequence of the downstream part of the pck gene (SEQ ID NO. 16) was amplified with P21 And P22 as primers. Using the purified PCR products described above as the upstream and downstream homologous arms of the pyc*-dapB operon, respectively, when they were integrated into the genome of Corynebacterium glutamicum ATCC13032, the purpose of knocking out pck could be achieved.

Using the pyc* gene with point mutation constructed in (2) and the genomic DNA of Corynebacterium glutamicum ATCC13032 as templates, primers were designed to amplify pyc* and dapB (SEQ ID NO. 17), respectively. The base information of related gene sequences was obtained from the NCBI database, and totally six pairs of primers were designed to construct the pyc*-dapB gene fragment (Table 3).

TABLE 3

| | | SEQ |
|---|---|---|
| | | ID |
| Primer | Base sequence | NO. |
| P13 | tctagagtcgacctgcaggcatgcaagctt | 18 |
| | ACCT GGCCCT CGATACCT C | |
| P14 | cctaggcctgtaaAGTTCACGCTTAAGAAC | 19 |
| | TGCTAAATAAC | |
| P15 | tgtgagtcgacatTAGAGTAATTATTCCTT | 20 |
| | TCAACAAGAG | |
| P16 | atctggagaagtaTGCGTTAAACTTGGCCA | 21 |
| | AATG | |
| P17 | tccgttctagggaTTAGGAAACGACGACGA | 22 |
| | TC | |
| P18 | aggaataattactctaAT GT CGACT C | 23 |
| | AC AC AT CTTC | |
| P19 | ttaagcgtgaactTTACAGGCCTAGG | 24 |
| | TAATG | |
| P20 | tcgtcgtcgtttcctaaTCCCTAGAACGGA | 25 |
| | ACAAAC | |
| P21 | caagtttaacgcaTACTTCTCCAGATTTTG | 26 |
| | TG | |
| P22 | cgttgtaaaacgacggccagtgccaagctt | 27 |
| | GCGAATACTTCAACACTTG | |
| P23 | taccttgggcaggtcgtggg | 28 |
| P24 | tgggagcgttgtgcgctcga | 29 |

Using the genomic DNA of Corynebacterium glutamicum ATCC13032 as a template, the genes with lengths of 750 bp, 244 bp, 3423 bp, 896 bp and 767 bp were amplified with P13 and P14, P15 and P16, P17 and P18, P19 and P20, P21 and P22, respectively. These genes were the sequence of the upstream part of the pck gene, the promoter sequence of the pyc gene (SEQ ID NO. 57), the sequence of the pyc* gene, the sequence of the dapB gene, and the sequence of the downstream part of the pck gene.

The purified PCR product was mixed with an E. coli cloning vector pK18mobsacB, ligated and assembled using NEbuilder (NEBuilder HiFi DNA Assembly Cloning Kit), and then transformed into E. coli DH5α; transformants were screened on LB plates containing kanamycin (50 μg/mL). After the subculture for three generations, the transformants were identified by colony PCR using P23 and P24 as primers; a plasmid was extracted from the transformant identified positive.

The positive plasmid was sent for sequencing. As a result, the plasmid was a recombinant plasmid obtained by inserting pyc*-dapB into the vector pK18mobsacB, and named YZ039, shown in FIG. 7.

The homologous recombinant plasmid YZ039 sequenced positive was electrotransformed into Corynebacterium glutamicum EPCG1007. Colonies with recombinant plasmids integrated into chromosomes were obtained by kanamycin resistance forward screening. Positive colonies with second homologous recombination were obtained by sucrose reverse screening. Using P23 and P24 as primers, PCR amplification and identification was carried out on the positive colonies to obtain a recombinant bacterium identified positive, named Corynebacterium glutamicum EPCG1009.

The genomic DNA of the recombinant bacterium was extracted and sequenced. The results confirmed that the pck gene in Corynebacterium glutamicum EPCG1007 had been successfully knocked out, the pyc*-dapB gene segment was also inserted, and Corynebacterium glutamicum EPCG1009 was successfully constructed.

(4) Increase in Copies of lysC, Ddh and lysA Genes

Primers were designed respectively according to the lysC (SEQ ID NO.30), ddh (SEQ ID NO.31), and ysA (SEQ ID NO.32) genes of Corynebacterium glutamicum ATCC13032 in Genbank and their upstream and downstream sequences.

Using the genomic DNA of Corynebacterium glutamicum ATCC13032 as a template, primers were designed to amplify lysC, ddh, and lysA genes respectively.

TABLE 4

| | | SEQ |
|---|---|---|
| | | ID |
| Primer | Base sequence | NO. |
| P25 | caggtcgactctagaggatccccggg | 33 |
| | AAAGGAGGACAACCATGGCcctggtcgtacag | |
| P26 | CACCGACATCATCTTCACCTGC | 34 |
| | gttgtcctcctttTTAGCGTCCGGTGCCTGC | |
| P27 | caccggacgctaaAAAGGAGGACAAC | 35 |
| | CATGACCAACATCCGCG | |
| P28 | gttgtcctcctttTTAGACGTCGCGTGCGATC | 36 |
| P29 | acgcgacgtctaaAAAGGAGGACA | 37 |
| | ACCATGGCTACAGTTGAAAAT | |
| P30 | ctcatccgccaaaacagccaagctgaattc | 38 |
| | TTATGCCTCTAGTGAGAGG | |

Using the genomic DNA of Corynebacterium glutamicum ATCC13032 as a template, the genes with lengths of 1266 bp, 963 bp and 1338 bp were amplified with P25 and P26, P27 and P28, P29 and P30.

The purified PCR product was mixed with an E. coli cloning vector pXMJ19, ligated and assembled using NEbuilder (NEBuilder HiFi DNA Assembly Cloning Kit), and then transformed into E. coli DH5α; transformants were screened on LB plates containing chloromycetin (20 μg/mL). After the subculture for three generations, the transformants were identified by colony PCR using P25 and P30 as primers; a plasmid was extracted from the transformant identified positive.

The positive plasmid was sent for sequencing. As a result, the plasmid was a recombinant plasmid obtained by inserting lysC, ddh, and lysA into the vector pXMJ19, and named YZ035, shown in FIG. 8.

The homologous recombinant plasmid YZ035 sequenced positive was electrotransformed into Corynebacterium glutamicum EPCG1009. The positive colonies that can grow on the resistant plate were identified by PCR amplification using P25 and P30 as primers to obtain the recombinant bacterium identified positive, named Corynebacterium glutamicum EPCG1010.

The genomic DNA of the recombinant bacterium was extracted and sequenced. The results confirmed that the free plasmid YZ035 was successfully introduced into Corynebacterium glutamicum EPCG1009, and Corynebacterium glutamicum EPCG1010 was successfully constructed.

### Example 2 Promoter Replacement of Asparaginase Encoding Gene NCgl2026 in Lysine Chassis Engineering Bacterium

Primers were designed respectively according to the upstream and downstream sequences of the NCg2026 gene promoter and P_{tuf} promoter sequence (SEQ ID NO. 40) of Corynebacterium glutamicum ATCC13032 in Genbank.

Using the genomic DNA of Corynebacterium glutamicum ATCC13032 as a template, the upstream homologous arm of the NCg2026 gene promoter was amplified by PCR with P31 and P32 as primers; the promoter P_{tuf} was amplified with P33 and P34 as primers; and the downstream homologous arm of the NCg2026 gene promoter was amplified with P35 and P36 as primers. Using the purified PCR product as a template and P31 and P36 as primers, SOE PCR was performed for amplification to obtain a 1800 bp PCR product, which is a segment containing upstream and downstream homologous arms of the replacement promoter Pₜa and the replaced promoter Pₜ.

The above 1800 bp PCR product was double-digested with Xba I and EcoR I, and then ligated with the double-digested homologous recombinant vector pK18mobsacB (purchased from ATCC, Cat. No. 87097). The ligation product was transformed into E. coli DH5α by chemical transformation, and the transformants were screened on LB plates containing kanamycin (50 μg/mL). After the subculture for three generations, the transformants were identified by colony PCR using P31 and P36 as primers to obtain a 1800 bp positive transformant; the plasmid was extracted from the transformant identified positive, and the plasmid was double digested by Xba I and EcoR I and identified; the obtained 1800 bp plasmid was positive.

The positive plasmid was sent for sequencing. As a result, the plasmid was a recombinant plasmid (shown in FIG. 2) obtained by inserting the strong promoter P_{tuf} containing upstream and downstream homologous arms into the vector pK18mobsacB, and named YZ022, shown in FIG. 2.

TABLE 6

| | | SEQ |
|---|---|---|
| | | ID |
| Primer | Base sequence | NO. |
| P31 | CCGGAATTCTGCTCAGGAGCAACAGTATT | 41 |
| P32 | CATTCGCAGGGTAACGGCCAGCGCTCTAGCGTATCAACTA | 42 |
| P33 | TAGTTGATACGCTAGAGCGCTGGCCGTTACCCTGCGAATG | 43 |
| P34 | GTGGAGTGCTGCTTCGACATTGTATGTCCTCCTGGACTTC | 44 |
| P35 | GAAGTCCAGGAGGACATACAATGTCGAAGCAGCACTCCAC | 45 |
| P36 | TGCTCTAGACAGCGATGGCAGCTTCCACC | 46 |

The homologous recombinant plasmid YZ022 sequenced positive was electrotransformed into Corynebacterium glutamicum EPCG1010. Colonies with recombinant plasmids integrated into chromosomes were obtained by kanamycin resistance forward screening. Positive colonies with second homologous recombination were obtained by sucrose reverse screening. Using P31 and P36 as primers, PCR amplification and identification was carried out on the positive colonies to obtain a 1800 bp recombinant bacterium, named Corynebacterium glutamicum EPCG1036.

The genomic DNA of the recombinant bacterium was extracted and sequenced. The results confirmed that the NCg2026 promoter in Corynebacterium glutamicum EPCG1010 was successfully replaced with the endogenous strong promoter P_{tuf} of Corynebacterium glutamicum, and Corynebacterium glutamicum EPCG1036 was successfully constructed.

### Example 3 Increase of Copies of Asparaginase Encoding Gene NCgl2026 in Lysine Chassis Engineering Bacterium

Primers were designed according to the NCg2026 gene of Corynebacterium glutamicum ATCC13032 in Genbank and its upstream and downstream sequences.

Using Corynebacterium glutamicum ATCC13032 genomic DNA as a template, the upstream sequence of the target insertion site was amplified by PCR with P37 and P38 as primers to function as the upstream homologous arm for the increase of copies of the NCg2026 gene; the NCgl2026 gene was amplified with P39 and P40 as primers; the downstream sequence of the target insertion site was amplified with P41 and P42 as primers to function as the downstream homologous arm for the increase of copies of the NCg2026 gene. Using the purified PCR product as a template and P37 and P42 as primers, SOE PCR was performed for amplification to obtain a 2778 bp PCR product, which is a segment containing the upstream and downstream homologous arms of the target insertion site and the NCg2026 gene.

The above 2778 bp PCR product was double-digested with Xba I and Nhe I, and then ligated with the double-digested homologous recombinant vector pK18mobsacB (purchased from ATCC, Cat. No. 87097). The ligation product was transformed into E. coli DH5α by chemical transformation, and the transformants were screened on LB plates containing kanamycin (50 μg/mL). After the subculture for three generations, transformants were identified by colony PCR using P37 and P42 as primers to obtain a 2778 bp positive transformant; a plasmid was extracted from the transformant identified positive, and the plasmid was double digested by Xba I and Nhe I and identified; the obtained 2778 bp plasmid was positive.

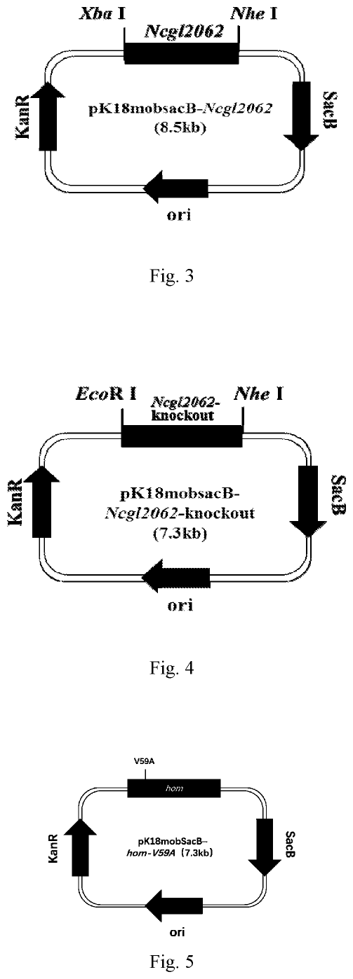

The positive plasmid was sent for sequencing. As a result, the plasmid was a recombinant plasmid (shown in FIG. 3) obtained by inserting the upstream and downstream homologous arms of the target insertion site and the NCg2026 gene into the vector pK18mobsacB, and named YZ023, shown in FIG. 3.

TABLE 7

| | | SEQ |
|---|---|---|
| | | ID |
| Primer | Base sequence | NO. |
| P37 | TGCTCTAGAAAGGGCAATGAGTTTGTCGA | 47 |
| P38 | GTGGAGTGCTGCTTCGACATTTAGTTCTCCAAGTAGAGCC | 48 |
| P39 | GGCTCTACTTGGAGAACTAAATGTCGAAGCAGCACTCCAC | 49 |
| P40 | TATCAGACGAGATCTTGGATTAGTAAAGCGTCACCGGAT | 50 |
| P41 | ATCCGGTGACGCTTTACTAATCCAAGATCTCGTCTGATA | 51 |
| P42 | CTAGCTAGCGTGTGGATCCGAGCGCGAAG | 52 |

The homologous recombinant plasmid YZ023 sequenced positive was electrotransformed into Corynebacterium glutamicum EPCG1010. Colonies with recombinant plasmids integrated into chromosomes were obtained by kanamycin resistance forward screening. Positive colonies with second homologous recombination were obtained by sucrose reverse screening. Using P37 and P42 as primers, PCR amplification and identification was carried out on the positive colonies to obtain a 2778 bp recombinant bacterium, named Corynebacterium glutamicum EPCG1039.

The genomic DNA of the recombinant bacterium was extracted and sequenced. The results confirmed that a copy of the NCg2026 gene has been successfully inserted at the target site in Corynebacterium glutamicum EPCG1010, and Corynebacterium glutamicum EPCG1039 has been successfully constructed.

### Example 4 Knockout of Asparaginase Encoding Gene NCg2026 from Lysine Chassis Engineering Bacterium

Primers were designed according to the NCg2026 gene of Corynebacterium glutamicum ATCC13032 in Genbank and its upstream and downstream sequences.

Using the genomic DNA of Corynebacterium glutamicum ATCC13032 as a template, the upstream homologous arm of the NCg2026 gene was amplified by PCR with P43 and P44 as primers; and the downstream homologous arm of the NCgl2026 gene was amplified with P45 and P46 as primers. Using the purified PCR product as a template and P43 and P46 as primers, SOE PCR was performed for amplification to obtain a 1600 bp PCR product, which is a segment containing the upstream and downstream homologous arms of the NCg2026 gene.

The above 1600 bp PCR product was double-digested with EcoR I and Nhe I, and then ligated with the double-digested homologous recombinant vector pK18mobsacB (purchased from ATCC, Cat. No. 87097). The ligation product was transformed into E. coli DH5α by chemical transformation, and the transformants were screened on LB plates containing kanamycin (50 μg/mL). After the subculture for three generations, transformants were identified by colony PCR using P43 and P46 as primers to obtain a 1600 bp positive transformant; a plasmid was extracted from the transformant identified positive, and the plasmid was double digested by EcoR I and Nhe I and identified; the obtained 1600 bp plasmid was positive.

The positive plasmid was sent for sequencing. As a result, the plasmid was a recombinant plasmid obtained by inserting the fragment containing the upstream and downstream homologous arms of the NCgl2026 gene into the vector pK18mobsacB, and named YZ025, shown in FIG. 4.

TABLE 8

| | | SEQ |
|---|---|---|
| | | ID |
| Primer | Base sequence | NO. |
| P43 | CCGGAATTCTGCTCAGGAGCAACAGTATT | 53 |
| P44 | ATGCAAGACCAAGGGCGAAAGCGCTCTAGCGTATCAACTA | 54 |
| P45 | TAGTTGATACGCTAGAGCGCTTTCGCCCTTGGTCTTGCAT | 55 |
| P46 | CTAGCTAGCTTATGAGGTAGGCGTGCAAT | 56 |

The homologous recombinant plasmid YZ025 sequenced positive was electrotransformed into Corynebacterium glutamicum EPCG1010. Colonies with recombinant plasmids integrated into chromosomes were obtained by kanamycin resistance forward screening. Positive colonies with second homologous recombination were obtained by sucrose reverse screening. Using P43 and P46 as primers, PCR amplification and identification was carried out on the positive colonies to obtain a 1600 bp recombinant bacterium, named Corynebacterium glutamicum EPCG1038.

The genomic DNA of the recombinant bacterium was extracted and sequenced. The results confirmed that the NCgl2026 gene was knocked out from Corynebacterium glutamicum EPCG1010, and Corynebacterium glutamicum EPCG1038 was successfully constructed.

### Example 5 Application of Lysine Engineering Bacteria of Corynebacterium glutamicum in Fermentation Production of Lysine

The L-lysine-producing Corynebacterium glutamicum EPCG1036, EPCG1038, and EPCG1039 constructed in Examples 2 to 4 and the original strain EPCG1010 were cultured at the shake flask level and the 3 L fermentor level respectively to produce L-lysine as follows.

(1) Shake Flask Fermentation:

Corynebacterium glutamicum EPCG1036, EPCG1038, EPCG1039 and EPCG1010 were inoculated in 500 ml Erlenmeyer flasks containing 50 ml of the seed medium described below, and cultured with shaking at 220 rpm for 8-9 h at 30° C. Then, 5 ml of each seed culture solution was inoculated into a 500 ml baffled bottle containing 50 ml of the fermentation medium described below, and cultured with shaking at 220 rpm for 42-46 h at 37° C. After fermentation for 6 h, isopropyl-β-D-thiogalactopyranoside (IPTG) with a final concentration of 1 mmol/L was added to induce the expression of the target gene. Concentrated ammonia water was intermittently supplemented to control the pH of the fermentation broth between 7.0 and 7.2. According to the residual sugar, glucose mother liquor with a concentration of 400 g/L was added to control the residual sugar of the fermentation broth at 5-10 g/L.

(2) 3 L Fermentor Fermentation:

Corynebacterium glutamicum EPCG1036, EPCG1038, EPCG1039 and EPCG1010 were inoculated in 1000 ml Erlenmeyer flasks containing 100 ml of the seed medium described below, and cultured with shaking at 220 rpm for 8-9 h at 30° C. Then, each seed culture solution was inoculated into a 3 L fermentor containing 900 ml of the fermentation medium described below, and cultured under the pressure of 0.01 MPa for 42-46 h at 37° C. The seed solution was inoculated at 10 vol % into a fermentation medium containing chloromycetin with a final concentration of 10 μg/ml. The fermentor used is a 3 L fermentor: equipped with a built-in constant-speed programmable control pump, which can achieve constant-speed feeding. During the fermentation process, 600 g/L glucose was supplemented by a peristaltic pump to control the concentration of glucose in the fermentation system at 5-10 g/L, and the fermentation temperature was maintained at 30° C. by virtue of a heating jacket and cooling water; the air was supplied to provide dissolved oxygen, and the rotation speed and dissolved oxygen signal were cascaded to control the dissolved oxygen at 30%; concentrated ammonia was supplemented to adjust the pH at about 6.9. The fermentation continued for 52 h. When OD₆₀₀=4-5, IPTG (isopropylthiogalactoside, the final concentration is 0.1 mmol/L) was added to induce expression of the gene carried by the recombinant plasmid.

The seed medium and fermentation medium are as follows:

Seed Medium (pH 7.0)

20 g of sucrose, 10 g of peptone, 5 g of yeast extract, 3.5 g of urea, 4 g of monopotassium phosphate, 10 g of dipotassium phosphate, 0.5 g of magnesium sulfate heptahydrate, 0.2 mg of biotin, 1.5 mg of vitamin B1, 2 mg of calcium dextrose, and 3 mg of nicotinamide (dissolved in 1 L of distilled water).

Fermentation Medium (pH 7.0)

40 g of glucose, 20 g of molasses, 0.4 g of phosphoric acid, 15 g of ammonium sulfate, 0.87 g of magnesium sulfate heptahydrate, 0.88 mg of biotin, 6.3 mg of vitamin B1, 6.3 mg of calcium dextropantothenate, and 42 mg of nicotinamide (dissolved in 1 L of distilled water).

(3) Detection of Lysine Production

Hplc Method:

1. Mobile Phase:

Organic phase:methanol:acetonitrile:water=45:45:10 (V/V);

Aqueous phase: 12.436 g of NaH₂PO₄.2H₂O is dissolved in 2 L of ultrapure water and the pH of the obtained solution is adjusted to 7.8 with NaOH.

2. Elution Procedure:

| Time (min) | Aqueous phase (%) | Organic phase (%) |
|---|---|---|
| 0.00 | 100.0 | 0.0 |
| 1.90 | 100.0 | 0.0 |
| 18.10 | 43.0 | 57.0 |
| 18.60 | 0.0 | 100.0 |
| 22.30 | 0.0 | 100.0 |
| 23.20 | 100.0 | 0.0 |
| 26.00 | 100.0 | 0.0 |

Solutions with standard concentration were prepared with a standard lysine product, the concentrations were 0.2 g/L, 0.4 g/L, 0.8 g/L, 1.6 g/L, and standard curves were plotted according to the peak area to calculate the concentrations of lysine in the fermentation broth as follows:

TABLE 9

| Lysine (g/L) | EPCG1036 | EPCG1038 | EPCG1039 | EPCG1010 |
|---|---|---|---|---|
| Shake flask | 10.60 ± 0.19 | 6.24 ± 0.11 | 12.93 ± 0.21 | 7.46 ± 0.35 |
| fermentation | | | | |
| 3 L Fermentor | 14.17 ± 0.49 | 8.96 ± 0.43 | 19.68 ± 0.66 | 12.34 ± 0.18 |
| fermentation | | | | |

The results of shake flask fermentation experiments showed that the expression of the asparaginase gene was enhanced, and the production of lysine was increased significantly; after knockout of the gene, the production of lysine was decreased significantly.

Corresponding to the results of the shake flask fermentation, when fermenting at the 3 L fermentor level, by increasing a copy of the asparaginase encoding gene, the production of lysine was increased by 59.48%; by replacing the asparaginase encoding gene promoter with a strong promoter, the production of lysine was increased by 14.83%; by knocking out the asparaginase encoding gene, the production of lysine was decreased by 27.39%.

### Example 6 Enhanced Expression of Asparaginase Encoding Gene NCgl2026 in Corynebacterium pekinense 1.563

Taking Corynebacterium pekinense AS1.563 capable of accumulating lysine as the original strain, the effect of the expression of the asparaginase encoding gene on the accumulation of lysine was analyzed.

(1) Strong Promoter Replacement of NCg2026 Gene

The recombinant vector YZ022 constructed in Example 2 was transformed into Corynebacterium pekinense AS1.563 (China Center of Industrial Culture Collection, CICC10178) to achieve the replacement of the NCg2026 gene promoter with the strong promoter P_{tuf}.

Colonies with recombinant plasmids integrated into chromosomes were obtained by kanamycin resistance forward screening. Positive colonies with second homologous recombination were obtained by sucrose reverse screening. Using P31 and P36 as primers, PCR amplification and identification was carried out on the positive colonies to obtain a 1800 bp recombinant bacterium, named Corynebacterium glutamicum CP1008.

The genomic DNA of the recombinant bacterium was extracted and sequenced. The results confirmed that the NCgl2026 promoter in Corynebacterium pekinense AS1.563 was successfully replaced with the endogenous strong promoter P_{tuf} of Corynebacterium glutamicum, and Corynebacterium glutamicum CP1008 was successfully constructed.

(2) Increase of Copies of NCg2026 Gene

The recombinant vector YZ023 constructed in Example 3 was transformed into Corynebacterium pekinense AS1.563 to increase copies of the NCgl2026 gene.

Colonies with recombinant plasmids integrated into chromosomes were obtained by kanamycin resistance forward screening. Positive colonies with second homologous recombination were obtained by sucrose reverse screening. Using P37 and P42 as primers, PCR amplification and identification was carried out on the positive colonies to obtain a 2778 bp recombinant bacterium, named Corynebacterium glutamicum CP1009.

The genomic DNA of the recombinant bacterium was extracted and sequenced. The results confirmed that a copy of the NCg2026 gene has been successfully inserted at the target site in Corynebacterium pekinense AS1.563, and Corynebacterium pekinense CP1009 has been successfully constructed.

(3) Knockout of Asparaginase Encoding Gene NCgl2026 from Corynebacterium pekinense AS1.563

The recombinant vector YZ025 constructed in Example 4 was transformed into Corynebacterium pekinense AS1.563 to knock out the NCg2026 gene.

Colonies with recombinant plasmids integrated into chromosomes were obtained by kanamycin resistance forward screening. Positive colonies with second homologous recombination were obtained by sucrose reverse screening. Using P43 and P46 as primers, PCR amplification and identification was carried out on the positive colonies to obtain a 1600 bp recombinant bacterium, named Corynebacterium glutamicum CP1010.

The genomic DNA of the recombinant bacterium was extracted and sequenced. The results confirmed that the NCgl2026 gene was knocked out from Corynebacterium pekinense AS1.563, and Corynebacterium pekinense CP1010 was successfully constructed.

### Example 7 Application of Lysine Engineering Bacteria of Corynebacterium pekinense in Fermentation Production of Lysine

The L-lysine-producing strains, Corynebacterium pekinense CP1008, CP1009 and CP1010 constructed in Example 6 and the original strain AS1.563 were cultured at the shake flask level and the 3 L fermentor level respectively to produce L-lysine as follows.

(1) Shake Flask Fermentation:

Corynebacterium pekinense CP1008, CP1009, CP1010 and AS1.563 were inoculated in 500 ml Erlenmeyer flasks containing 50 ml of the seed medium described below, and cultured with shaking at 220 rpm for 8-9 h at 30° C. Then, 5 ml of each seed culture solution was inoculated into a 500 ml baffled bottle containing 50 ml of the fermentation medium described below, and cultured with shaking at 220 rpm for 42-46 h at 37° C. Concentrated ammonia water was intermittently supplemented to control the pH of the fermentation broth between 7.0 and 7.2. According to the residual sugar, glucose mother liquor with a concentration of 400 g/L was added to control the residual sugar of the fermentation broth at 5-10 g/L.

(2) 3 L Fermentor Fermentation:

Corynebacterium pekinense CP1008, CP1009, CP1010 and AS1.563 were inoculated in 1000 ml Erlenmeyer flasks containing 100 ml of the seed medium described below, and cultured with shaking at 220 rpm for 8-9 h at 30° C. Then, each seed culture solution was inoculated into a 3 L fermentor containing 900 ml of the fermentation medium described below, and cultured under the pressure of 0.01 MPa for 42-46 h at 37° C. The fermentor used is a 3 L fermentor: equipped with a built-in constant-speed programmable control pump, which can achieve constant-speed feeding. During the fermentation process, 600 g/L glucose was supplemented by a peristaltic pump to control the concentration of glucose in the fermentation system at 5-10 g/L, and the fermentation temperature was maintained at 30° C. by virtue of a heating jacket and cooling water; the air was supplied to provide dissolved oxygen, and the rotation speed and dissolved oxygen signal were cascaded to control the dissolved oxygen at 30%; concentrated ammonia was supplemented to adjust the pH at about 6.9. The fermentation continued for 52 h.

The seed medium and fermentation medium are as follows:

Seed Medium (pH 7.0)

20 g of sucrose, 10 g of peptone, 5 g of yeast extract, 3.5 g of urea, 4 g of monopotassium phosphate, 10 g of dipotassium phosphate, 0.5 g of magnesium sulfate heptahydrate, 0.2 mg of biotin, 1.5 mg of vitamin B1, 2 mg of calcium dextrose, and 3 mg of nicotinamide (dissolved in 1 L of distilled water).

Production of Medium: pH7.0

40 g of glucose, 20 g of molasses, 0.4 g of phosphoric acid, 15 g of ammonium sulfate, 0.87 g of magnesium sulfate heptahydrate, 0.88 mg of biotin, 6.3 mg of vitamin B1, 6.3 mg of calcium dextropantothenate, and 42 mg of nicotinamide (dissolved in 1 L of distilled water).

After the cultivation was completed, HPLC analysis was performed to determine the content of L-lysine produced by the strains. The concentrations of L-lysine in Corynebacterium pekinense CP1008, CP1009, CP1010 and AS1.563 cultures were shown in Table 10.

TABLE 10

| Lysine (g/L) | EPCG1008 | EPCG1009 | EPCG1010 | AS1.563 |
|---|---|---|---|---|
| Shake flask | 21.23 ± 0.28 | 20.40 ± 0.15 | 9.27 ± 0.37 | 13.98 ± 0.81 |
| fermentation | | | | |
| 3 L Fermentor | 39.65 ± 2.23 | 28.98 ± 1.43 | 14.98 ± 0.56 | 21.26 ± 1.31 |
| fermentation | | | | |

As can be seen from the table above, the transformed strains showed significant differences, both at the shake flask level and at the 3 L fermentor level.

The difference trend between shake flask fermentation and fermentor fermentation remains the same. That is, after the expression of asparaginase gene was enhanced, the production of lysine was increased. Correspondingly, after the gene was knocked out, the production of lysine was decreased significantly.

In terms of fermenter acid production data, compared with AS1.563, CP1008's lysine production was increased by 86.6%; compared with AS1.563, CP1009's lysine production was increased by 36.3%. Compared with AS1.563, CP1010's lysine production was decreased by 29.5%.

It should be noted that the above-described examples are merely illustrative of the invention and are not intended to limit the implementations. Other variations or modifications of the various forms may be made by those skilled in the art in light of the above description. There is no need and no way to exhaust all of the implementations. Obvious changes or variations resulting therefrom are still within the scope of the invention.

1. A recombinant bacterium capable of producing L-lysine, wherein the recombinant bacterium has increased expression and/or activity of asparaginase compared to an original bacterium, and the original bacterium refers to a strain capable of accumulating lysine.

2. The recombinant bacterium according to claim 1, wherein the recombinant bacterium has at least two copies of asparaginase encoding gene, and/or the expression of the asparaginase encoding gene of the recombinant bacterium is mediated by a regulatory element with high transcription or high expression activity;
preferably, the regulatory element is a strong promoter;
more preferably, the strong promoter is a P_{tuf} promoter.

3. The recombinant bacterium according to claim 1, wherein the recombinant bacterium has reduced expression and/or activity of homoserine dehydrogenase compared to the original bacterium;
preferably, the reduced expression of homoserine dehydrogenase is achieved in at least one of the following ways: (A) the homoserine dehydrogenase encoding gene of the recombinant bacterium is inactivated, and (B) the expression of the homoserine dehydrogenase encoding gene of the recombinant bacterium is mediated by a regulatory element with low transcription or low expression activity;
the reduced activity of homoserine dehydrogenase is achieved by mutating the 59th valine of homoserine dehydrogenase of the recombinant bacterium to alanine, preferably, the omoserine dehydrogenase encoding gene of the recombinant bacterium is SEQ ID NO.1.

4. The recombinant bacterium according to claim 1, wherein the recombinant bacterium has increased expression and/or activity of pyruvate carboxylase compared to the original bacterium;
preferably, the increased expression of pyruvate carboxylase is achieved by at least one of the following ways: (C) the recombinant bacterium has at least two copies of pyruvate carboxylase encoding gene, and (D) the expression of the pyruvate carboxylase encoding gene of the recombinant bacterium is mediated by a regulatory element with high transcription or high expression activity;
the increased activity of pyruvate carboxylase is achieved by mutating the 458th proline of the pyruvate carboxylase of the recombinant bacterium to serine, preferably, the pyruvate carboxylase encoding gene of the recombinant bacterium is SEQ ID NO.8.

5. The recombinant bacterium according to claim 1, wherein the recombinant bacterium has reduced expression and/or activity of phosphoenolpyruvate carboxykinase compared to the original bacterium; preferably, the phosphoenolpyruvate carboxykinase encoding gene of the recombinant bacteria is inactivated, and/or the expression of the phosphoenolpyruvate carboxykinase encoding gene is mediated by a regulatory element with low transcription or low expression activity;
more preferably, the inactivated is knocking out the phosphoenolpyruvate carboxykinase encoding gene of the recombinant bacterium.

6. The recombinant bacterium according to claim 1, wherein the recombinant bacterium has increased expression and/or activity of dihydropyridine dicarboxylate reductase (dapB) compared to the original bacterium;
preferably, the recombinant bacterium has at least two copies of dihydropyridine dicarboxylate reductase encoding gene, and/or the expression of the dihydropyridine dicarboxylate reductase encoding gene is mediated by a regulatory element with high transcription or high expression activity;
more preferably, the regulatory element is a strong promoter;
most preferably, the strong promoter is a P_{tuf} promoter of the original bacterium.

7. The recombinant bacterium according to claim 1, wherein the recombinant bacterium has increased expression and/or activity of aspartate kinase, diaminopimelate dehydrogenase and/or diaminopimelate decarboxylase compared to the original bacterium;
preferably, the recombinant bacterium has at least two copies of aspartate kinase encoding gene, diaminopimelate dehydrogenase encoding gene and/or diaminopimelate decarboxylase encoding gene, and/or the expression of the aspartate kinase encoding gene, the diaminopimelate dehydrogenase encoding gene and/or the diaminopimelate decarboxylase encoding gene is mediated by a regulatory element with high transcription or high expression activity;
more preferably, the regulatory element is a strong promoter;
most preferably, the strong promoter is a P_{tuf} promoter of the original bacterium.

8. The recombinant bacterium according to claim 1, wherein the original bacterium is a bacterium selected from Corynebacterium, Brevibacterium, Bacillus, Bifidobacterium, and Lactobacillus or a fungus selected from yeast;
preferably, the bacterium of Corynebacterium is selected from Corynebacterium glutamicum, Corynebacterium pekinense, Corynebacterium efficiens, Corynebacterium crenatum, Corynebacterium thermoaminogenes, Corynebacterium aminogenes, Corynebacterium lilium, Corynebacterium callunae, and Corynebacterium herculis;
the bacterium of Brevibacterium is selected from Brevibacteriaceae fivum, Brevibacteriaceae lactofermentum and Brevibacteriaceae ammoniagenes;
the bacterium of Bacillus is selected from Bacillus licheniformis, Bacillus subtilis and Bacillus pumilus;
the bacterium of Bifidobacterium is selected from Bifidobacterium bifidum, Bifidobacterium longum, Bifidobacterium breve, and Bifidobacterium adolescentis;
the bacterium of Lactobacillus is one of Lactobacillus acidophilus, Lactobacillus casei, Lactobacillus delbrueckii subsp and Lactobacillus fermentum;
the fungus of yeast is selected from Candida utilis, Saccharomyces cerevisiae, Pichia pastoris and Hansenula polymorpha.

9. A construction method of the recombinant bacterium according to claim 1, comprising the following step:
increasing the expression and/or activity of asparaginase in an original bacterium,
wherein preferably, the increasing the expression and/or activity of the asparaginase in the original bacterium is achieved by at least one of: (E) increasing the copy number of asparaginase encoding gene in the original bacterium, and (F) replacing a regulatory element for the asparaginase encoding gene in the original bacterium with a regulatory element with high transcription or high expression activity.

10. A production method of L-lysine, comprising the following step: fermenting and culturing the recombinant bacterium according to claim 1.


SEQUENCE LISTING (57 sequences)

SEQ ID NO: 1 - DNA, 1638 bp, Corynebacterium glutamicum
ctgcgacagcatggaactcagtgcaatggctgtaaggcctgcaccaacaatgattgagcgaagctccaaaatgtcctccccgggttgatattagatttcataaatatactaaaaatcttgagagtttttccgttgaaaactaaaaagctgggaaggtgaatcgaatttcggggctttaaagcaaaaatgaacagcttggtctatagtggctaggtaccctttttgttttggacacatgtagggtggccgaaacaaagtaataggacaacaacgctcgaccgcgattatttttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgcttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatctccctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaa

SEQ ID NO: 2 - DNA, 26 bp, Artificial Sequence
  hom-V59A upstream forward primer
  primer_bind (1)..(26)
gctctagaagctgtttcacaatttct

SEQ ID NO: 3 - DNA, 19 bp, Artificial Sequence
  hom-V59A mutation site reverse primer
  primer_bind (1)..(19)
atatcagaagcagcaatgc

SEQ ID NO: 4 - DNA, 19 bp, Artificial Sequence
  hom-V59A mutation site forward primer
  primer_bind (1)..(19)
gcattgctgcttctgatat

SEQ ID NO: 5 - DNA, 27 bp, Artificial Sequence
  hom-V59A downstream reverse primer
  primer_bind (1)..(27)
ccggaattcccaacaacttgatggtgt

SEQ ID NO: 6 - DNA, 19 bp, Artificial Sequence
  hom-V59A forward identified primer
  primer_bind (1)..(19)
tctacgttgtatctcgcac

SEQ ID NO: 7 - DNA, 18 bp, Artificial Sequence
  hom-V59A reverse identifiedprimer
  primer_bind (1)..(18)
caggcgaccagctgcttc

SEQ ID NO: 8 - DNA, 2280 bp, Corynebacterium glutamicum
aaaaccgatgtttgattgggggaatcgggggttacgatactaggacgcagtgactgctatcacccttggcggtctcttgttgaaaggaataattactctagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcactcgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagct

SEQ ID NO: 9 - DNA, 28 bp, Artificial Sequence
  pyc-P458S upstream forward primer
  primer_bind (1)..(28)
cccaagctttgactgctcactgcagcgt

SEQ ID NO: 10 - DNA, 17 bp, Artificial Sequence
  pyc-P458S mutation site reverse primer
  primer_bind (1)..(17)
aggtgcgagtgatcggc

SEQ ID NO: 11 - DNA, 17 bp, Artificial Sequence
  pyc-P458S mutation site forward primer
  primer_bind (1)..(17)
gccgatcactcgcacct

SEQ ID NO: 12 - DNA, 26 bp, Artificial Sequence
  pyc-P458S downstream reverse primer
  primer_bind (1)..(26)
gctctagagcgtcgattgctggacgc

SEQ ID NO: 13 - DNA, 19 bp, Artificial Sequence
  pyc-P458S forward identified primer
  primer_bind (1)..(19)
cgcaaattagcaacagaag

SEQ ID NO: 14 - DNA, 18 bp, Artificial Sequence
  pyc-P458S reverse identified primer
  primer_bind (1)..(18)
ccttaatggccaagatgt

SEQ ID NO: 15 - DNA, 750 bp, Corynebacterium glutamicum
acctggccctcgatacctcgagtctggtggcggctttgtctgaagatatttctggcgccggattaaatgacctgaaagttctcgacgtcggcggcggacccggatacttcgccgaagcctttgagacactgggcgccacctacttctccgtcgaacccgacgttggcgaaatgtccgcagctggcatcgacgtccacggatcagtccgcggatccggcctcgacctgccgtttcttcccgattcctttgacgtggtgtactcctccaacgttgcagaacatgtctccgcaccgtgggaattgggagaagaaatgctccgcgtcacccgcagcggcggcctggcaatcctgagctacaccatttggttagggcccttcggcggccatgaaaccggactgtgggaacactacgttggcggagaatttgcccgcgatcgctacacgaagaaacacgggcacccgcctaagaacgttttcggggagtcactgtttaatgtgtcctgccgggaggggctggaatggggagcctccgtgggcaatgcggaattggttgccgcttttccccgctaccacccgtattgggtctggtggatggttaaagtcccagtgctccgagaattcgcggtaagtaacttggtgttggtgtttaaaaagcactgaggttttgaggaattcatcgcttaacgacaagaaaggctcccactttcggtgggagcctttcttgttatttagcagttcttaagcgtgaact

SEQ ID NO: 16 - DNA, 767 bp, Corynebacterium glutamicum
tacttctccagattttgtgtcattcgacagagttctcgccccctagcgtagctttcagatacagaactagttaaaactttaggtgagacaacggacacatttgtcattaccagtggacctaccccctgcccacacgcatctacacactttctttaatatgagagcacccgtttaaatagcctattttgggggtggtttcaagaattaacctcaaccgttctccgacagttcattccccgtccatggccattgggttcagatttgggcaattctcacacattccaggggacaactttcccagttttcccaccattaacacttaacattcggacaataggcaacaaaacgccaagaacagcggtaatggtaatccctttcccttaccctgccatcacaatccaagcactccgctagtggccgaccagcacaaaccggcccactgtcagtacacacctttttaaaacaacatttacactcacatgcatgcccgcactgtcaccacccgccctcaactaccgaactaaagatatgtacttgaagccaaatttttaccctagatcccccttttaaatactttgaaaattactcacacacatccccacgttaccccaaaggttatatccagttagtcgtatcaaaaagtgctctgatcttaactttgccctacctaaatacatgaccccaccacgacggccagtactaacgacagaatccactagcgaacccatttattaacaaacattgcaaacaagtgttgaagtattcgc

SEQ ID NO: 17 - DNA, 960 bp, Corynebacterium glutamicum
gctccttttaaaaaattccacccgctgctgaaatgagcctttacaggcctaggtaatgctcaagtcctacgactaggcctgggtgctgtgcaatgttgcgcacacccaccaagacacctggtgcaaatgagttgcgatcataggagtcctgcttgatggtcaaggtctgaccctgggtgccaaagataacttgctcgtgagcaaccatgccggacatgcggactgcatgaaccgggattccatctacgcttgcgccacgggaaccctcaagtgcctgctcggtcgcatctggctgtgcgtccatgcctgcttctttgcgtgccgcagcaatgccctgagcagtgtggatcgcggtgcctgaaggtgcatccagcttgttggggtggtgcagctcaataacttcagctgattcgaagaagcgggcagcctgcttggaaaagaccatggtcaacaccgcagagatagcaaagttaggtgcgatcagaacaccgacattgtcttttccttcaagccagtcgcgaacctgctccaaacgagcatcatcgaagcccgtggttccaacaaccgcagaaatgccgttgttgatgcagaactccaggttgcccatcacagcgttaggagtggtgaagtcaacgacaacttcagcgccgttgtctaccagaaggctcaaatcatcgtcgacgccgatctctgcaacaagctccagatcgtcggactcattgactgctgccacaatagtttgaccaacacggcctttggctccgagaacgccaaccttgattcccattatgctccttcattttcgtggggcgaagagtttttcaaacgcccaccagtctagacgtacccgttcagaaggtgctgatatccatacctaactgaccgttttggtctgtgttgttaacgattgttcatcagtttgttccgttctagggattaatcacacaggtggaactatgt

SEQ ID NO: 18 - DNA, 49 bp, Artificial Sequence
  pck upstream homologous arms forward primer
  primer_bind (1)..(49)
tctagagtcgacctgcaggcatgcaagcttacctggccctcgatacctc

SEQ ID NO: 19 - DNA, 41 bp, Artificial Sequence
  pck upstream homologous arms reverse primer
  primer_bind (1)..(41)
cctaggcctgtaaagttcacgcttaagaactgctaaataac

SEQ ID NO: 20 - DNA, 40 bp, Artificial Sequence
  pyc promoter forward primer
  primer_bind (1)..(40)
tgtgagtcgacattagagtaattattcctttcaacaagag

SEQ ID NO: 21 - DNA, 34 bp, Artificial Sequence
  pyc promoter reverse primer
  primer_bind (1)..(34)
atctggagaagtatgcgttaaacttggccaaatg

SEQ ID NO: 22 - DNA, 32 bp, Artificial Sequence
  pyc* forward primer
  primer_bind (1)..(32)
tccgttctagggattaggaaacgacgacgatc

SEQ ID NO: 23 - DNA, 36 bp, Artificial Sequence
  pyc* reverse primer
  primer_bind (1)..(36)
aggaataattactctaatgtcgactcacacatcttc

SEQ ID NO: 24 - DNA, 31 bp, Artificial Sequence
  dapB forward primer
  primer_bind (1)..(31)
ttaagcgtgaactttacaggcctaggtaatg

SEQ ID NO: 25 - DNA, 36 bp, Artificial Sequence
  dapB reverse primer
  primer_bind (1)..(36)
tcgtcgtcgtttcctaatccctagaacggaacaaac

SEQ ID NO: 26 - DNA, 32 bp, Artificial Sequence
  pck downstream homologous arms forward primer
  primer_bind (1)..(32)
caagtttaacgcatacttctccagattttgtg

SEQ ID NO: 27 - DNA, 49 bp, Artificial Sequence
  pck downstream homologous arms reverse primer
  primer_bind (1)..(49)
cgttgtaaaacgacggccagtgccaagcttgcgaatacttcaacacttg

SEQ ID NO: 28 - DNA, 20 bp, Artificial Sequence
  pyc*-dapB identified forward primer
  primer_bind (1)..(20)
taccttgggcaggtcgtggg

SEQ ID NO: 29 - DNA, 20 bp, Artificial Sequence
  pyc*-dapB identified reverse primer
  primer_bind (1)..(20)
tgggagcgttgtgcgctcga

SEQ ID NO: 30 - DNA, 1266 bp, Corynebacterium glutamicum
atggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcatcttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaa

SEQ ID NO: 31 - DNA, 963 bp, Corynebacterium glutamicum
atgaccaacatccgcgtagctatcgtgggctacggaaacctgggacgcagcgtcgaaaagcttattgccaagcagcccgacatggaccttgtaggaatcttctcgcgccgggccaccctcgacacaaagacgccagtctttgatgtcgccgacgtggacaagcacgccgacgacgtggacgtgctgttcctgtgcatgggctccgccaccgacatccctgagcaggcaccaaagttcgcgcagttcgcctgcaccgtagacacctacgacaaccaccgcgacatcccacgccaccgccaggtcatgaacgaagccgccaccgcagccggcaacgttgcactggtctctaccggctgggatccaggaatgttctccatcaaccgcgtctacgcagcggcagtcttagccgagcaccagcagcacaccttctggggcccaggtttgtcacagggccactccgatgctttgcgacgcatccctggcgttcaaaaggcagtccagtacaccctcccatccgaagacgccctggaaaaggcccgccgcggcgaagccggcgaccttaccggaaagcaaacccacaagcgccaatgcttcgtggttgccgacgcggccgatcacgagcgcatcgaaaacgacatccgcaccatgcctgattacttcgttggctacgaagtcgaagtcaacttcatcgacgaagcaaccttcgactccgagcacaccggcatgccacacggtggccacgtgattaccaccggcgacaccggtggcttcaaccacaccgtggaatacatcctcaagctggaccgaaacccagatttcaccgcttcctcacagatcgctttcggtcgcgcagctcaccgcatgaagcagcagggccaaagcggagctttcaccgtcctcgaagttgctccatacctgctctccccagagaacttggacgatctgatcgcacgcgacgtctaa

SEQ ID NO: 32 - DNA, 1338 bp, Corynebacterium glutamicum
atggctacagttgaaaatttcaatgaacttcccgcacacgtatggccacgcaatgccgtgcgccaagaagacggcgttgtcaccgtcgctggtgtgcctctgcctgacctcgctgaagaatacggaaccccactgttcgtagtcgacgaggacgatttccgttcccgctgtcgcgacatggctaccgcattcggtggaccaggcaatgtgcactacgcatctaaagcgttcctgaccaagaccattgcacgttgggttgatgaagaggggctggcactggacattgcatccatcaacgaactgggcattgccctggccgctggtttccccgccagccgtatcaccgcgcacggcaacaacaaaggcgtagagttcctgcgcgcgttggttcaaaacggtgtgggacacgtggtgctggactccgcacaggaactagaactgttggattacgttgccgctggtgaaggcaagattcaggacgtgttgatccgcgtaaagccaggcatcgaagcacacacccacgagttcatcgccactagccacgaagaccagaagttcggattctccctggcatccggttccgcattcgaagcagcaaaagccgccaacaacgcagaaaacctgaacctggttggcctgcactgccacgttggttcccaggtgttcgacgccgaaggcttcaagctggcagcagaacgcgtgttgggcctgtactcacagatccacagcgaactgggcgttgcccttcctgaactggatctcggtggcggatacggcattgcctataccgcagctgaagaaccactcaacgtcgcagaagttgcctccgacctgctcaccgcagtcggaaaaatggcagcggaactaggcatcgacgcaccaaccgtgcttgttgagcccggccgcgctatcgcaggcccctccaccgtgaccatctacgaagtcggcaccaccaaagacgtccacgtagacgacgacaaaacccgccgttacatcgccgtggacggaggcatgtccgacaacatccgcccagcactctacggctccgaatacgacgcccgcgtagtatcccgcttcgccgaaggagacccagtaagcacccgcatcgtgggctcccactgcgaatccggcgatatcctgatcaacgatgaaatctacccatctgacatcaccagcggcgacttccttgcactcgcagccaccggcgcatactgctacgccatgagctcccgctacaacgccttcacacggcccgccgtcgtgtccgtccgcgctggcagctcccgcctcatgctgcgccgcgaaacgctcgacgacatcctctcactagaggcataa

SEQ ID NO: 33 - DNA, 58 bp, Artificial Sequence
  lysC forward primer
  primer_bind (1)..(58)
caggtcgactctagaggatccccgggaaaggaggacaaccatggccctggtcgtacag

SEQ ID NO: 34 - DNA, 53 bp, Artificial Sequence
  lysC reverse primer
  primer_bind (1)..(53)
caccgacatcatcttcacctgcgttgtcctcctttttagcgtccggtgcctgc

SEQ ID NO: 35 - DNA, 43 bp, Artificial Sequence
  ddh forward primer
  primer_bind (1)..(43)
caccggacgctaaaaaggaggacaaccatgaccaacatccgcg

SEQ ID NO: 36 - DNA, 32 bp, Artificial Sequence
  ddh reverse primer
  primer_bind (1)..(32)
gttgtcctcctttttagacgtcgcgtgcgatc

SEQ ID NO: 37 - DNA, 45 bp, Artificial Sequence
  lysA forward primer
  primer_bind (1)..(45)
acgcgacgtctaaaaaggaggacaaccatggctacagttgaaaat

SEQ ID NO: 38 - DNA, 49 bp, Artificial Sequence
  lysA reverse primer
  primer_bind (1)..(49)
ctcatccgccaaaacagccaagctgaattcttatgcctctagtgagagg

SEQ ID NO: 39 - DNA, 978 bp, Corynebacterium glutamicum
atgtcgaagcagcactccacaccattaaacaatgatgaagaacacacttccgctcctcaaaaggttgcggtaatcaccacgggcggaaccatcgcctgtacttccgacgcaaatgggcatctgcttcccaccgtcagcggtgcagacctgcttgcgccaatcgcaccacggttcaatggagcgcagatcgctttcgaaatccacgaaatcaaccgccttgattcctcctccatgacgtttgaggatctcgattccatcatcgccacggttcataaggtgttggaggatccggatgttgttggcgtagtagttacccacggcaccgattccatggaagagtccgccatcgccgtagacaccttccttgatgatccccgcccagtcattttcaccggcgcccaaaaacccttcgatcatcccgaagccgacggcccaaacaaccttttcgaagcctgcctcatcgcatccgacccctccgctcgcggaattggtgcactcattgtcttcggtcacgccgtcatccctgctcgcggctgcgttaaatggcacacctctgatgagctggcgtttgcaaccaacggccctgaagaaccagagcgccccgatgcgctgcccgtagctaaattggcggatgtctctgtcgaaatcatccccgcataccctggtgccaccggcgcaatggtggaagctgccatcgctgccggtgctcaaggacttgtagtggaagcaatgggatcaggcaatgttggttcccgcatgggtgatgccctaggtaaagcacttgacgctggaattcccgtggtgatgagcactagggttcctcgtggtgaagtatccggagtgtatggcggtgcaggtggaggtgcgactttggctgcgaagggcgctgtgggatctcgctacttcagagctggtcaggcacgtattttgctcgcgattgccattgcgacgggcgcacatccggtgacgctttactaa

SEQ ID NO: 40 - DNA, 200 bp, Corynebacterium glutamicum
tggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 41 - DNA, 29 bp, Artificial Sequence
  insert Ptuf upstream homologous arms forward primer
  primer_bind (1)..(29)
ccggaattctgctcaggagcaacagtatt

SEQ ID NO: 42 - DNA, 40 bp, Artificial Sequence
  insert Ptuf upstream homologous arms reverse primer
  primer_bind (1)..(40)
cattcgcagggtaacggccagcgctctagcgtatcaacta

SEQ ID NO: 43 - DNA, 40 bp, Artificial Sequence
  Ptuf forward primer
  primer_bind (1)..(40)
tagttgatacgctagagcgctggccgttaccctgcgaatg

SEQ ID NO: 44 - DNA, 40 bp, Artificial Sequence
  Ptuf reverse primer
  primer_bind (1)..(40)
gtggagtgctgcttcgacattgtatgtcctcctggacttc

SEQ ID NO: 45 - DNA, 40 bp, Artificial Sequence
  insert Ptuf downstream homologous arms forward primer
  primer_bind (1)..(40)
gaagtccaggaggacatacaatgtcgaagcagcactccac

SEQ ID NO: 46 - DNA, 29 bp, Artificial Sequence
  insert Ptuf downstream homologous arms reverse primer
  primer_bind (1)..(29)
tgctctagacagcgatggcagcttccacc

SEQ ID NO: 47 - DNA, 29 bp, Artificial Sequence
  increase ncgl2062 copy upstream homologous arms forward primer
  primer_bind (1)..(29)
tgctctagaaagggcaatgagtttgtcga

SEQ ID NO: 48 - DNA, 40 bp, Artificial Sequence
  increase ncgl2062 copy upstream homologous arms reverse primer
  primer_bind (1)..(40)
gtggagtgctgcttcgacatttagttctccaagtagagcc

SEQ ID NO: 49 - DNA, 40 bp, Artificial Sequence
  ncgl2062 forward primer
  primer_bind (1)..(40)
ggctctacttggagaactaaatgtcgaagcagcactccac

SEQ ID NO: 50 - DNA, 39 bp, Artificial Sequence
  ncgl2062 reverse primer
  primer_bind (1)..(39)
tatcagacgagatcttggattagtaaagcgtcaccggat

SEQ ID NO: 51 - DNA, 39 bp, Artificial Sequence
  increase ncgl2062 copy downstream homologous arms forward primer
  primer_bind (1)..(39)
atccggtgacgctttactaatccaagatctcgtctgata

SEQ ID NO: 52 - DNA, 29 bp, Artificial Sequence
  increase ncgl2062 copy downstream homologous arms reverse primer
  primer_bind (1)..(29)
ctagctagcgtgtggatccgagcgcgaag

SEQ ID NO: 53 - DNA, 29 bp, Artificial Sequence
  ncgl2062 knockout upstream homologous arms forward primer
  primer_bind (1)..(29)
ccggaattctgctcaggagcaacagtatt

SEQ ID NO: 54 - DNA, 40 bp, Artificial Sequence
  ncgl2062 knockout upstream homologous arms reverse primer
  primer_bind (1)..(40)
atgcaagaccaagggcgaaagcgctctagcgtatcaacta

SEQ ID NO: 55 - DNA, 40 bp, Artificial Sequence
  ncgl2062 knockout downstream homologous arms forward primer
  primer_bind (1)..(40)
tagttgatacgctagagcgctttcgcccttggtcttgcat

SEQ ID NO: 56 - DNA, 29 bp, Artificial Sequence
  ncgl2062 knockout downstream homologous arms reverse primer
  primer_bind (1)..(29)
ctagctagcttatgaggtaggcgtgcaat

SEQ ID NO: 57 - DNA, 244 bp, Corynebacterium glutamicum
tgcgttaaacttggccaaatgtggcaacctttgcaaggtgaaaaactggggcggggttagatcctggggggtttatttcattcactttggcttgaagtcgtgcaggtcaggggagtgttgcccgaaaacattgagaggaaaacaaaaaccgatgtttgattgggggaatcgtgtggtataatggtaggacgcagtgactgctatcacccttggcggtctcttgttgaaaggaataattactcta